(19) United States
(12) Patent Application Publication
(10) Pub. No.: US 2026/0345940 A1
(43) Pub. Date: Nov. 5, 2026

(54) SYSTEM FOR SUPPLYING GASES OR GAS MIXTURES WITH FEEDING OF SUBSTANCES

(71) Applicant: Drägerwerk AG & Co. KGaA, Lübeck (DE)

(72) Inventors: Henning GERDER, Lübeck (DE); Christian BRENDLE, Lübeck (DE); Marco MONNIG, Lübeck (DE)

(21) Appl. No.: 19/742,116

(22) Filed: Jul. 15, 2026

Related U.S. Application Data
(63) Continuation of application No. 17/318,370, filed on May 12, 2021, now abandoned.

(30) Foreign Application Priority Data
May 13, 2020 (DE) 10 2020 112 951.3
Jan. 6, 2021 (DE) 10 2021 100 090.4

(51) Int. Cl. A61M 16/06; A61B 5/00; A61B 5/08; A61M 1/16; A61M 1/36; A61M 16/00; A61M 16/08; A61M 16/16; A61M 16/20; A61M 16/22

(52) U.S. Cl. CPC A61M 16/0672; A61B 5/0036; A61B 5/0044; A61B 5/08; A61M 1/1601; A61M 1/1698; A61M 1/3627; A61M 16/024; A61M 16/0891; A61M 16/16; A61M 16/205; A61M 16/22; A61M 1/3623; A61M 2202/0208; A61M 2202/0225; A61M 2202/0241; A61M 2205/75; A61M 2230/00

(57) ABSTRACT

A system (1000) for a feeding of substances to a patient (30) with ventilation and oxygenation of the patient. The system (1000) includes a ventilation system (1), an oxygenation system (2), a breathing gas dispensing path (3), a purge gas dispensing path (4), a breathing gas connection system (5), an oxygenation connection system (6), a dispensing system (7), a switching unit (8), and at least one control unit (9). The switching unit (8) is configured for a distribution or splitting of a quantity of a drug or anesthetic active ingredient, which quantity was dispensed into a gas mixture by means of the dispensing system (7), between the ventilation system (1) and the oxygenation system (2). The at least one control unit (9) is configured to control the switching unit (8).

### CROSS REFERENCE TO RELATED APPLICATIONS

This application is a continuation application of and claims the benefit of priority under 35 U.S.C. §120 of United States Application 17/318,370 filed May 12, 2021, which claims the benefit of priority under 35 U.S.C. §119 of German Applications 10 2020 112 951.3, filed May 13, 2020 and 10 2021 100 090.4, filed Jan. 6, 2021, the entire contents of each application are incorporated herein by reference.

### TECHNICAL FIELD

The invention pertains to a combined system with a device for an extracorporeal membrane oxygenation of a patient and with a device for carrying out a ventilation of a patient with feeding of substances in breathing gases and/or breathing gas mixtures to the patient. The system according to the present invention with the devices for ventilation and for extracorporeal membrane oxygenation makes it possible to feed inhalable substances or anesthetics by means of the gas or gas mixture supplied to the patient. The substances are anesthetics, anesthesia gases, narcotics, which are, for example and preferably, dissolved in the gas phase or vapor phase, drug active ingredients or drugs dissolved in the gas phase or vapor phase, which are suitable for administration by inhalation into the breathing gas. The term "breathing gas" is defined below in the sense of the present invention as a generic term for gas quantities fed to the patient or removed from the patient, so that inhaled gas, exhaled gas, breathing gases, inhaled gases, exhaled gases as well as breathing gas, breathing gases are defined by it.

### BACKGROUND

The application of conventional ventilation in intensive care units as well as during the carrying out of an operation often lead to undesired side effects, for example, barotrauma/volutrauma and aspiration, which may cause damage to the lungs in some cases and may lead to such complications as pneumonia or sepsis. To avoid further damage and as treatment in the case of damaged heart or lungs, there are approaches to oxygenation and circulatory assist, such as venovenous extracorporeal membrane oxygenation (v.v. ECMO), pumpless extracorporeal lung assist for the removal of carbon dioxide (pECLA) and veno-arterial extracorporeal membrane oxygenation (v.a. ECMO).

Anesthesia devices and ventilators, which can be used either for carrying out surgical procedures in operating rooms (OR) or for ventilating in an intensive care unit (ICU), are known from the state of the art.

US 2016067434 A1 discloses a ventilator for a ventilation of a patient for a use in an intensive care unit. The ventilator shown shall be used for the goal of avoiding complications during the carrying out of the ventilation.

US 6155256 A discloses an anesthesia device with a fresh gas system for a mixing of gases from at least two gas sources, which has a vaporizer for a feeding of anesthetic into the fresh gas.

US 4148312 A discloses a combination of the anesthesia device and a ventilator. Unconsciousness, insensitivity to pain and relaxing of muscles of the patient are essential for carrying out a ventilation during a surgical procedure. For this purpose, different volatile anesthetics (halothane, isoflurane, desflurane, sevoflurane, ether) as well as nitrous oxide with different hypnotic, analgesic and muscle-relaxing properties combined with air and oxygen are fed to the patient by inhalation by means of the anesthesia device, for example, by means of an endotracheal tube. In addition, drugs are also usually administered into the blood circulation invasively. The addition of the volatile anesthetics into the breathing gas or into the breathing gas mixture may take place, for example, by means of evaporation with an anesthetic vaporizer - also called an anesthetic vaporizer or vaporizer.

US 2016008567 A1 discloses a system for dispensing narcotics or volatile anesthetics.

WO 09033462 A1 discloses an anesthetic evaporator with a storage tank and with a feeding and dispensing device, wherein a vapor pressure of the anesthetic is generated by elevated temperature and a saturated anesthetic vapor is generated.

So-called heart-lung machines (HLM) are used especially when performing operations on the heart. These heart-lung machines (HLM) take over the function of the heart and lungs for the duration of the surgical procedure, i.e., the feeding of oxygen into the blood circulation of the patient and the removal of carbon dioxide from the blood circulation of the patient as well as the flow of blood into the blood vessels.

GB 2568813 A1 discloses a heart-lung machine for extracorporeal gas exchange and oxygenation.

US 2020038564 A1 discloses a blood pump, which is suitable for an extracorporeal transport of blood.

US 9901885 B2 discloses a membrane, which is configured and intended for a blood-to-gas and gas-to-blood exchange.

US 6174728 B1, US 4279775 A and US 2003064525 A1 disclose devices for a determination of components and blood gases in the blood of living beings.

### SUMMARY

With knowledge of the above-mentioned state of the art, an object of the present invention is to provide a system, which makes it possible to feed substances in the gas form into the breathing circuit and to feed substances in the gas form into the blood circulation of the patient outside of the body.

The system according to the present invention makes possible, for example, for an application in the clinical setting of anesthesia, a simultaneous and/or parallel coordinated operation with the feeding of anesthesia gases into the breathing system via the ventilation tubes within the framework of an anesthesia by inhalation to the patient and with the feeding of the anesthesia gases via a gas/blood exchange system (membrane oxygenation, ECMO, oxygenator). Likewise, both an administration of volatile anesthetics via the lungs into the cardiovascular system of the patient, but also an administration of volatile anesthetics via the gas/blood exchange system into the blood circulation of the patient (extracorporeal circulation) are made possible using the system according to the present invention.

A system according to the present invention has
- a ventilation system,
- an oxygenation system,
- a dispensing system,
- a switching unit,
- a breathing gas dispensing path,
- a purge gas dispensing path,
- a breathing gas connection system,
- an oxygenation connection system, and
- at least one control unit.

The ventilation system is configured for supplying breathing gases or breathing gas mixtures to the patient. In its usual configuration the ventilation system is part of an anesthesia device or ventilator. Anesthesia devices and ventilators have devices for supplying, feeding and removing breathing gases or breathing gas mixtures and substances to and from the patient, e.g., devices for gas mixing and for gas feeding, for example, a gas feed unit (blower, piston drive), as well as devices for gas carrying, such as a breathing gas connection system, for example, in the form of ventilation tubes and a connection element - the so-called Y-piece - for the connection of the ventilation tubes to an endotracheal tube, to a breathing mask or to a tracheostoma. In addition, connection elements that also comprise an exhalation valve are also known.

In addition, anesthesia devices and/or ventilators likewise have elements for the measurement-based detection of defined and/or set pressures, flow rates and other operating parameters of a mechanical ventilation with feed of gases and gas mixtures. For mechanical ventilation, at least the following parameters are set and/or monitored, such as inspiratory as well as expiratory ventilation pressures, ventilation rate, inspiration-to-expiration ratio, pressure upper and lower limits, flow rate upper and lower limits, volume upper and lower limits and gas concentrations. In the sense of the present invention, a ventilation system supports the system in the case of object and function to ensure the ventilation of the lungs, i.e., to ensure collapsing of the lungs or of individual lung regions (alveoli). In addition, the ventilation system supports the patient during the O₂/CO₂ exchange in the lungs.

The breathing gas connection system is configured for a gas-carrying connection for supplying with feeding and removal of quantities of breathing gases or breathing gas mixtures to the patient.

The oxygenation connection system is configured for a fluidic connection to the blood circulation for the feeding and removal of quantities of blood of the patient.

The enriched breathing gas or breathing gas mixture supplied by the ventilation system is fed to the patient by means of the breathing gas connection system as a fresh breathing gas mixture using an inspiratory ventilation tube. The breathing gas or breathing gas mixture exhaled by the patient is fed back or removed by means of the breathing gas connection system by an expiratory ventilation tube.

The at least one control unit is configured according to the present invention for the control of the switching unit. The control includes here the coordination of the splitting and/or distribution of gas quantities supplied and/or fed by the dispensing system between the dispensing paths, i.e., between the purge gas dispensing path and the breathing gas dispensing path. The control unit carries out, as it were, a gas or gas mixture management between the two dispensing paths.

The gas quantities supplied and/or fed by the dispensing system are enriched or saturated in or by the dispensing system with quantities of substances, with quantities of volatile substances or with quantities of volatile anesthetics. With the control and/or coordination of the gas or gas mixture management, specifications are made that are suitable for at least one control unit as to what quantities of substances, volatile substances or volatile anesthetics are fed to the ventilation system and thus quantities of breathing gas also then fed into the breathing circuit and into the lungs of the patient from the ventilation system via the breathing gas ventilation system and thus then also fed or exchanged quantities of blood from the oxygenation system via the oxygenation connection system into the blood circulation of the patient.

Due to the control and coordination of the switching unit by means of the at least one control unit, especially of the control unit of the switching unit, for example, a setting of a balance between an anesthesia by inhalation and an extracorporeal anesthesia is possible, or else the elimination of this balance during the treatment is possible for medical reasons. The control unit in the switching unit can thus put into practice the specifications of a user with regard to the setting of or changes in a therapy focus related to the balance between extracorporeal anesthesia by means of the oxygenation system (ECMO, HLM) or anesthesia by inhalation by means of the ventilation system (anesthesia device) during the operation of the system according to the present invention, which makes it possible to feed substances in the gas form into the breathing circuit and to feed substances in gas form into the blood circulation of a patient outside of the body.

In one preferred embodiment, the exhaled breathing gas or breathing gas mixture that is fed back in the ventilation system is fed back into the fresh breathing gas mixture via a breathing gas absorber unit and subsequently again to the patient via the inspiratory ventilation tube. This recirculating system according to this preferred embodiment is designated as a so-called closed circuit system. The breathing gas absorber unit removes the carbon dioxide component from the exhaled breathing gas mixture, so that quantities of volatile anesthetic not absorbed by the patient are used again for the treatment after removal of carbon dioxide in the closed circuit.

The breathing gas absorber unit contains a special kind of lime granules (soda lime) known as breathing lime, usually consisting of calcium hydroxide [Ca(OH)₂] and/or sodium hydroxide [NaOH]. The carbon dioxide component is removed from the exhaled gas mixture by means of a chemical reaction with the release of heat and water. A waste gas outlet (waste), via which used quantities of exhaled breathing gas or breathing gas mixture can be fed for disposal, is provided in the ventilation system.

The switching unit makes it possible according to the present invention to switch, split or distribute gas quantities of fresh gas and substances, for example, volatile anesthetics or drugs, by means of the breathing gas dispensing path in the direction of the breathing system and by means of the purge gas dispensing path in the direction of the oxygenation system.

The oxygenation system is configured for supplying oxygen and for eliminating carbon dioxide in a blood circulation to the patient. The oxygenation system has a membrane for a gas/blood exchange. A quantity of oxygen is introduced by means of this membrane by means of a purge gas into the quantity of blood of the blood circulation of the patient and a quantity of carbon dioxide is removed from the blood circulation of the patient. The purge gas is supplied by the switching unit by means of the purge gas connection path to the oxygenation system.

In a preferred embodiment, the transport of the quantity of blood to the patient and from the patient can be carried out by blood feed devices, for example, by a blood feed unit (pump). Such a blood feed unit (pump) is preferably arranged in or at the oxygenation connection system or in or at the oxygenation system and is used for the transport of a quantity of blood to the patient and away from the patient. Such a pump may be coupled venovenously (VV-ECMO) or arteriovenously (VA-ECMO) by means of suitable infusion cannulas and tubes with typical external diameters in the range of about 3.0 mm to 12.0 mm. In this case, the pump feeds blood flow rates in the range of 0.2 L/min to 10 L/min to the oxygenation system and back again. Here as well, the blood circulation of the patient is accessed, for example, via the femoral artery and via the femoral vein, also via the femoral artery and the external jugular vein, as an alternative.

The blood feed unit makes it possible, especially in an embodiment of a blood feed unit that can be set in terms of the feed quantity, to carry out the extracorporeal blood-gas exchange in regard to the removal of carbon dioxide and the feeding of oxygen in a manner coordinated individually with the situation and with the patient.

The transport of the quantity of blood to the patient and from the patient may take place in a special embodiment without external blood supply. The quantity of blood is transported to the patient and away from the patient in such an embodiment by the pumping capacity of the heart of the patient himself. This is called pumpless extracorporeal membrane oxygenation or pumpless extracorporeal lung assist (pECLA). The coupling of the pumpless extracorporeal membrane oxygenation takes place arteriovenously, for example, by means of the femoral artery and femoral vein by means of suitable infusion cannulas and tubes with typical internal diameters in the range of about 3 mm to 7 mm, so that the heart typically feeds a blood flow rate in the range of 2 L/min to 2.5 L/min to the oxygenation system outside of the body and back again. The blood supplied by the oxygenation system and enriched with oxygen is fed invasively with a supply line by means of the oxygenation connection system to the patient as a quantity of blood that is fresh and enriched with oxygen, a quantity of blood enriched with carbon dioxide is fed back away from the patient by means of the oxygenation connection system to the oxygenation system. The oxygenation connection system thus makes it possible to supply the patient with quantities of blood enriched with volatile substances and with oxygen (O₂) and to remove quantities of blood enriched with carbon dioxide (CO₂).

In a preferred embodiment of the system, the dispensing system is configured for the dispensing of volatile substances. This other preferred embodiment offers the advantage that the administration of volatile agents is made possible with the system by means of the ventilation system; thus, for example, an anesthesia by inhalation can be carried out with it repeated. However, the administration by inhalation of quantities of volatile drugs may also be carried out by means of the ventilation system. In addition, an administration of volatile agents, for example, the administration of volatile anesthetics or volatile drugs is made possible with the system by means of the oxygenation system.

In a preferred embodiment of the system, the dispensing system is configured for the dispensing of volatile anesthetics. This other preferred embodiment offers the advantage that an anesthesia by inhalation can be carried out via the lungs as well as extracorporeally using the system combined with the ventilation system and the oxygenation system.

Preferred embodiments of the system according to the present invention may have configurations of the control unit as a central control system or as a central control unit. These other preferred embodiments offer the advantages that a variety of information can be processed centrally, can be compared to one another and then the checking, control and/or regulation of the ventilation, of the extracorporeal blood-gas exchange or of the carrying out of the anesthesia can be coordinated and controlled centrally. Changes in the modes of operation or treatment can now advantageously be coordinated centrally, for example, a setting of a balance between an anesthesia by inhalation and an extracorporeal anesthesia or even the elimination of this balance during the treatment for medical reasons with the establishing of a new treatment focus, e.g., essentially extracorporeal anesthesia or anesthesia by inhalation.

However, preferred embodiments of the system according to the present invention may likewise be configured with a plurality of individual control units, which in combination and interaction with one another then form a common control of the system. The control of the system may likewise be configured as a so-called "master-slave" arrangement by means of a plurality of control units (slave) in interaction with a central control unit (master). Control units may be arranged in the ventilation system, in the dispensing system, in the oxygenation system, in the switching unit or even in an external module. These preferred embodiments of the system offer as advantages the fact that data of various systems can be combined with one another, which also makes possible the combinations of devices of different manufacturers, and they make possible expansions of existing devices with other devices or modules. The coordination and cooperation with one another is made possible by means of coordinated protocol in the data exchange, for example, in a data network (LAN, WLAN).

In other preferred embodiments of the system, an individual control unit at least in the switching unit and/or an individual control unit in the dispensing unit and/or an external control unit can be arranged in the non-central control system

One or more of the individual control units and/or the external control unit may be configured for a control of the switching unit and/or of the dispensing unit. In this case, the control may comprise a coordination of the splitting and/or distribution of gas quantities that are supplied and/or fed by the dispensing system between the dispensing paths, i.e., between the purge gas dispensing path and the breathing gas dispensing path by means of the switching unit. In addition, the control may also comprise the manner of the dispensing by means of the dispensing unit, as well as a control of the switching unit and the dispensing system, which is combined and coordinated with the operation of the system for a supply of gas or gas mixtures with feeding of substances, for example, for carrying out an anesthesia by inhalation with combined feeding of anesthesia gases into the breathing circuit and into the blood circulation of a patient.

This other preferred embodiment offers the advantage that coordination and control of the system can be configured with regard to the requirements on computing power, storage requirement and response time given for the individual functions such that, for example, rule procedures with high performance requirements in terms of time for the dispensing can take place directly in a control unit in the dispensing system, but, for example, a switching of the splitting of the quantities of fresh gas to the oxygenation system and to the ventilation system can take place by means of the external control unit with moderate performance requirements in terms of time. Changes in this splitting of quantities could be made, for example, by a mobile terminal, for example, by a tablet computer, by a smartphone, or by a mobile phone connected in a wireless manner.

In another preferred embodiment of the system, at least one of the control units can take into account respective data of the ventilation system and/or of the oxygenation system provided in the control of the switching unit.

This other preferred embodiment offers the advantage that, for example, the information about changes which the user is making or which he has recently activated or initiated, for example, in the kind of ventilation at the ventilation system, can be taken into consideration in the control of the switching unit such that the implementation of the initiated changes is being waited for before a status change is made by the switching unit. The same applies to initiated changes at the oxygenation system in regard to the control of the switching unit. Moreover, possible alarms from the ventilation system and the oxygenation system can be taken into consideration for the control of the switching unit, for example, such that only defined changes in the operating state of the switching unit are possible in the presence of alarms.

The control unit or the individual control units is/are configured for a control of the switching unit and of the dispensing system. The control unit may also be configured for the control of the ventilation system, of the dispensing system, as well as of the oxygenation system. The control unit may in this case be arranged as a functional element or control module in or at the ventilation system, in or at the dispensing system, in or at the oxygenation system or be associated with the ventilation system, with the dispensing system, with the oxygenation system. As functional elements, the control unit as well as the individual control units provide a variety of functions for the operation of the system according to the present invention. A memory (RAM, ROM), which is configured for storage of a program code, is usually provided in the control unit. The running of the program code is coordinated by means of a microcontroller that is arranged as an essential element in the control unit or other embodiment of computing elements (FPGA, ASIC, μP, μC, GAL). The control unit and/or the individual control units are configured, prepared and intended to coordinate the operation of the system and/or the interaction of the ventilation system, the dispensing system, the oxygenation system and the switching unit and other components and systems and to carry out comparison operations, computing operations, storage and data organization of the data quantities, actuations of actuators and sensors, acquisition of measured values of sensors, data and information processing, as well as provision of the information and data to components in the interior of the system and to the outside of the system, which are necessary in the process.

For an application in the clinical area of anesthesia these volatile anesthetics are fed to the breathing gas or breathing gas mixture by means of the dispensing system to the ventilation system and enter the bronchial tract and lungs of the patient via the ventilation tubes, Y-piece and endotracheal tube, or breathing mask or tracheostoma with the breathing gases or breathing gas mixtures. A mixing of fed gases, such as oxygen, air, nitrous oxide with a fresh gas mixture

(FG) is carried out by means of the dispensing system with subsequent adding of volatile anesthetics or of other substances for further use in the ventilation system or in the oxygenation system. The dispensing system is configured for the dispensing of volatile anesthetics or other substances, for example, drugs. The following list includes some examples of possibilities - drug active ingredients which are possibly also soluble in the gas phase or vapor phase - such as substances or agents for having an effect on the cardiovascular system, e.g., with effect on the blood pressure and heart rate, drugs for having an effect on the metabolism, on the liquid balance or on the hormonal situation of the patient as well as drugs, which can be fed by inhalation in the gas phase or in the vapor phase in regard to function and/or cure, or recovery or for pain treatment as therapeutic actions for organs, e.g., lungs, heart, kidneys, pancreas, liver, stomach, intestines, sex organs, sensory organs, brain, nervous system, bronchial tract, skeleton, skin, and muscles, thyroid, gallbladder. Volatile anesthetics or narcotics can be added, for example, by means of so-called vaporizers. Vaporizers operate according to the dispensing principle of a change in flow rate ratios between a main stream and a side stream. The main stream and the side stream are merged at the outlet of the vaporizer. A saturation of the fed fresh gas (FG) with the volatile anesthetics or with the other substances takes place in the side stream; the degree of the addition, and thus also the concentration, of volatile anesthetics or other substances can be set at the outlet of the vaporizer by an adjustment or setting of the flow rate ratios between the main stream and the side stream. Thus, an enrichment of the fresh gas consisting of oxygen, nitrogen and air mixed with volatile anesthetics or with other substances or drugs takes place in the dispensing system. In the embodiment of the dispensing system in the area of anesthesia, the

switching unit is arranged downstream of the dispensing system in the gas stream; the switching unit may in this case be configured as a component of the dispensing system.

A switching, splitting or distribution of gas quantities of the fresh gas between the breathing system and the oxygenation system takes place by means of the switching unit according to the present invention. A feed with splitting or distribution of the volatile anesthetics or of the other substances by the dispensing system towards the breathing system and/or towards the oxygenation system also takes place indirectly with the switching, splitting or distribution of gas quantities of the fresh gas. Suitable devices for switching and distribution are, for example, valves or valve arrays, 3/2-way valves or a combination of two 2/2-way valves arranged parallel in the gas flow with corresponding state control for distribution and splitting into partial quantities by the dispensing system by means of the breathing gas dispensing path towards the breathing system, or by means of the purge gas dispensing path towards the oxygenation system. The connection between the switching unit to the breathing system with feeding of a partial quantity of the enriched fresh gas to the breathing system takes place by means of the breathing gas dispensing path. The connection between the switching unit to the oxygenation system with feeding of a partial quantity of the enriched fresh gas to the oxygenation system takes place by means of the purge gas dispensing path. The switching unit is configured for a switching between the two dispensing paths and in interaction with the two dispensing paths for the distribution and splitting of the enriched fresh gas quantity to the oxygenation system and to the breathing system.

In an alternative embodiment for an application in the clinical area of intensive care, these volatile anesthetics or other substances are fed to the breathing gas or breathing gas mixtures by means of the dispensing system and enter the bronchial tract and lungs of the patient with the breathing gases or breathing gas mixtures via the ventilation tubes, Y-piece and endotracheal tube or the breathing mask. This other preferred embodiment offers the advantage that an administration by inhalation of quantities of volatile drugs can be carried out by means of the ventilation system. In addition, an administration of volatile drugs is made possible with the system by means of the oxygenation system.

In another preferred embodiment of the system, a purge gas absorber unit and/or an additional gas feed unit, for example, configured as a blower may be arranged in the oxygenation system or in the purge gas dispensing path. This other preferred embodiment offers the advantage that purge gas prepared by means of the purge gas absorber unit can be fed back into the purge gas dispensing path and can subsequently enter the blood circulation of the patient at the membrane again by means of the oxygenation connection system. The purge gas absorber unit removes the carbon dioxide component, delivered from the blood circulation of the patient, from the purge gas, so that quantities of volatile anesthetic not absorbed by the patient can be used again in the closed circuit for treatment. The additional gas feed unit makes possible a circulation of the purge gas in a cycle. Thus, it can be avoided that purge gas enriched with volatile anesthetic has to be removed as used gas by means of a waste gas outlet directly after flowing past the membrane once and thus valuable anesthetic cannot be used again for the further treatment. Such an additional gas feed unit may be arranged in combination with the additional purge gas absorber unit as a module, for example, as a type of plug-in module in the oxygenation system. The additional gas feed unit and the purge gas absorber unit may be configured together or even separately as independent units or modules, which can be connected to the oxygenation system, for example, as external modules. Thus, the purge gas absorber unit is advantageously configured to remove a portion of carbon dioxide from the purge gas, so that quantities of volatile anesthetic that are not introduced into the blood circulation at the membrane can be used again in the closed circuit in the operation of the oxygenation system after removal of carbon dioxide. The purge gas absorber unit of the oxygenation system is configured similarly to the breathing gas absorber unit of the ventilation system, it contains lime granules (soda lime), usually consisting of calcium hydroxide [Ca(OH) ₂] and/or sodium hydroxide [NaOH]. The carbon dioxide component is removed from the purge gas by means of a chemical reaction with the release of heat and water. A waste gas outlet (waste), via which used quantities of purge gas can be fed for disposal, is provided in the oxygenation system. All used quantities of gas are usually introduced into the infrastructure of the hospital from the anesthesia device by means of an anesthesia gas scavenging system (AGS: Anesthesia Gas Scavenger) and correspondingly disposed of properly.

The quantities of gas fed to the process gas analysis units are introduced into the infrastructure of the hospital and disposed of after the analysis in most cases likewise by means of the anesthesia gas scavenging system. In some cases, these analyzed quantities of gas may, however, also be reused and may again be fed, for example, in/at the absorber units into the ventilation system. Configurations with an open anesthesia gas scavenging (ORS: Open Reservoir Scavenger) are also possible; in this case, the used anesthesia gas in the exhaled gas or breathing gas mixture is filtered by means of an activated carbon collector and retained and subsequently the filtered exhaled gas or exhaled gas mixture is fed to the room air.

In preferred embodiments of the system, one or more process gas analysis units (PGA) can be arranged in the system or associated with the system for an analysis of gases, gas mixtures, liquids and/or quantities of blood. These process gas analysis units take gas samples from the gas stream with a suctioning feed and by means of a measured gas line at the patient and/or at the ventilation system and/or at the oxygenation system and then analyze the gas samples for the concentrations of sought substances, for example, anesthesia gases, nitrous oxide, carbon dioxide, and oxygen. Such process gas analysis units may provide data and/or information determined based on the analysis to the control unit and/or to the control system or to individual control units.

These other preferred embodiments offer the advantage that it is possible to continuously monitor the functions of the dosages of the volatile substances and anesthetics during the operation, and the effect of dosages and/or dosage changes on the patient or on the state of the patient can be estimated based on this.

Some exemplary possibilities for the arrangement, association and use of process gas analysis units (PGA) in the system will be explained in more detail below.

The process gas analysis units (PGA) can be arranged at individual components in the system and thus be used for analysis independently of one another in special embodiments.

However, it is also possible and in the sense of the present invention as alternative other embodiments also covered that a process gas analysis unit (PGA-C) arranged centrally in the system with an additional switching and distribution control unit, for example, configured as controllable and/or controlled valve arrays forms a kind of analysis center. In this case, corresponding gas samples are provided to the central process gas analysis unit (PGA-C) by the ventilation system, by the oxygenation system or by the dispensing system or by the switching unit by means of the switching and distribution control unit and then analyzed in series one after the other as needed by the central process gas analysis unit (PGA-C). The switching and distribution control unit is to be fed to the central process gas analysis unit (PGA-C) by devices for the switching, distribution and feeding of gas samples of the individual components in the system, especially from the oxygenation system, oxygenation connection system, dispensing system, switching unit, connection element located close to the patient and to provide for an analysis and to coordinate the feeding of gas samples. This other preferred embodiment offers the advantage that a respective process gas analysis unit (PGA) does not have to be arranged at each unit or at each module of the system. This may reduce the design and operating effort related to the components, such as sensor system, power supply, interfaces and operating software and simplify the function and cooperation especially in an embodiment with a central control unit. The results of the analysis can then be correspondingly provided to the individual control units or to the central control unit in a non-central or central manner. In one special embodiment, a blood gas analysis unit may also be integrated into the process gas analysis unit (PGA-C), which is arranged centrally in the system. Thus, such a process gas analysis unit may

be arranged in or at the ventilation system or in or at the breathing gas connection system or be associated with the ventilation system or with the breathing gas connection system for an analysis of breathing gases or breathing gas mixtures. This process gas analysis unit (PGA-VS) may provide data determined on the basis of the analysis to the control unit and/or to an individual control unit. In the breathing gas or breathing gas mixture the concentrations of defined gases can be determined by means of the process gas analysis unit, the knowledge of which is relevant for carrying out a ventilation or anesthesia. Knowledge with regard to concentrations of carbon dioxide and oxygen in the breathing gas mixture is relevant for carrying out a ventilation as well as carrying out an anesthesia. In addition, knowledge with regard to the concentrations of nitrous oxide and different anesthetics, for example, halothane, isoflurane, desflurane, sevoflurane or ether in the breathing gas mixture is relevant for carrying out an anesthesia. An additional such process gas analysis unit may be arranged in or at the oxygenation system or in or at the oxygenation connection system or be associated with the oxygenation system or with the oxygenation connection system for an analysis of purge gases. This additional process gas analysis unit (PGA-OS) may provide data determined based on the analysis to the control unit and/or to an individual control unit. Knowledge with regard to the concentrations of carbon dioxide and/or oxygen in the purge gas is relevant for carrying out an extracorporeal membrane oxygenation.

A special mode of configuration of the process gas analysis unit may be configured in a preferred embodiment as a configuration of a blood gas analysis unit (BGA) for an analysis of quantities of blood. This blood gas analysis unit according to this preferred embodiment may be arranged in or at the oxygenation system or in or at the oxygenation connection system or be associated with the oxygenation system or with the oxygenation connection system. The blood gas analysis unit (BGA) makes possible an analysis of the gases or gas mixtures dissolved in the blood of the patient, so that the blood gas analysis unit (BGA) has, for example, knowledge regarding a gas distribution (partial pressure) of O₂ (oxygen), CO₂ (carbon dioxide) as well as the pH value and the acid-base balance in the blood. The blood gas analysis unit may provide data determined on the basis of the analysis to the control unit and/or to an individual control unit. A knowledge of these values may often be interesting or relevant for an evaluation of the effect of anesthesia, ventilation and/or extracorporeal membrane oxygenation. This other preferred embodiment offers the advantage to utilize the knowledge of gas distribution (partial pressure) of O₂ (oxygen), CO₂ (carbon dioxide) for the monitoring of the control of the oxygenation system. In addition, information which is meaningful to the user with regard to the general state of the patient and in relation to the carrying out of the treatment may be provided by means of the values obtained regarding the acid-base balance and the pH value in the blood. In addition, this blood gas analysis unit (BGA) may also check and/or monitor the function of the oxygenation system (oxygenator quality) in the course of its use. Thus, information about the current state as well as about possible future changes in state or about changes in properties of the oxygenator or membrane can then be provided to the user in a timely manner.

The function of the oxygenation system may be impaired, for example, by coagulation effects (coagulation, clotting). Such a blood gas analysis unit (BGA) may be arranged in the oxygenation system in combination with the process gas analysis unit (PGA-OS) as a module, for example, as a type of plug-in module. The blood gas analysis unit (BGA) and the process gas analysis unit (PGA-OS) may be configured together or separately also as independent units or modules, which can be connected, for example, as external modules to the oxygenation system. This combination and configuration as a module, especially and for example as a plug-in module offers the advantage that the oxygenation system can be equipped with modules selectively and in a manner adapted to the situation, so that before the use the oxygenation system can be correspondingly set up with modules for blood gas analysis (BGA) and/or process gas analysis.

In a preferred embodiment of the system, a process gas analysis unit is arranged in or at the dispensing system or is associated with the dispensing system for an analysis. This process gas analysis unit (PGA-DS) may, in a manner similar to the process gas analysis unit, which is arranged at the ventilation system for analysis, carry out a gas analysis with regard to the concentrations of defined gases and especially determine the concentrations of nitrous oxide and different anesthetics, for example, halothane, isoflurane, desflurane, sevoflurane or ether, as well as oxygen in the fresh gas and provide data determined based on this analysis to the control unit and/or to an individual control unit. This other preferred embodiment offers the advantage that the composition of the fresh gas (FG) with concentrations of anesthetics, oxygen and nitrous oxide is continuously known during the operation and a control and monitoring, as well as a regulation of the dispensing of anesthetic, oxygen and nitrous oxide are made possible in the control unit in the dispensing system itself or in a central control unit.

In a preferred embodiment of the system, a process gas analysis unit is arranged in or at the switching unit or is associated with the switching unit for an analysis. This process gas analysis unit (PGA-US) may, in a manner similar to the process gas analysis unit, which is arranged at the dispensing system for analysis, carry out a gas analysis with regard to the concentrations of defined gases in the breathing gas dispensing path and/or in the purge gas dispensing path and especially determine the concentrations of nitrous oxide and different anesthetics, for example, halothane, isoflurane, desflurane, sevoflurane or ether, as well as oxygen in the fresh gas and provide data determined on the basis of this analysis to the control unit and/or to an individual control unit. This other preferred embodiment offers the advantage that the composition of the fresh gas (FG) with concentrations of anesthesia, oxygen and nitrous oxide is continuously known during the operation and a control with regard to the splitting of the fresh gas (FG) in the breathing gas dispensing path and in the purge gas dispensing path, including data related to concentrations of anesthetics, oxygen and/or nitrous oxide in the control unit, in the switching unit, in the dispensing system or in a central control unit is made possible.

In a preferred embodiment of the system, a breathing gas absorber unit is arranged in the breathing gas connection system and/or in the ventilation system for the removal of carbon dioxide from the breathing gases. This preferred embodiment offers the advantage that a portion of the anesthetic exhaled with the breathing gas mixture by exhalation can again be refed to the patient by inhalation, since the exhaled quantity of carbon dioxide is removed from the exhaled gas mixture by means of the breathing gas absorber. This makes possible an economical use of anesthetics and a lesser load on the environment with anesthetics.

In addition to a preferred embodiment with an additional gas inlet for the feeding of oxygen to the switching unit, an additional advantageous aspect is obtained with the arrangement of a process gas analysis unit (PGA-US) at the switching unit.

This process gas analysis unit (PGA-US), which is arranged in or at the switching unit or associated with the switching unit, can thus be used to determine the additional quantity of oxygen in the purge gas dispensing path, which quantity was introduced via the additional gas inlet, or to determine and monitor the concentration of oxygen in the purge gas dispensing path compared to the concentration of oxygen in the breathing gas dispensing path or compared to the concentration of oxygen in the fresh gas and to provide same to the control unit, to the individual control units or control modules by means of the data lines or data links. Especially in a configuration of the system with a central control unit, this central control unit is capable by means of these data of controlling the switching unit and thus of setting the gas concentrations in the purge gas dispensing path and in the breathing gas dispensing path. For this purpose, a corresponding additional switching unit or valve, which makes possible a controlled dispensing of oxygen from the additional gas inlet into the purge gas dispensing path, is provided in this configuration of this preferred embodiment with the additional gas inlet.

Data and/or information can be provided by means of data lines or data links in the system between the process gas analysis units, the control unit, the individual control units, configured, for example, as control modules. The data lines or data links are preferably configured as a wired or wireless data network (Ethernet, LAN, WLAN, Bluetooth, PAN) or bus system (CAN, LON), which has data nodes for data coordination (switch, hub, router), on the one hand, as well as components (database, server, router, access point) for data storage, data distribution. For example, a databank system can thus be connected to the data network for organization of patient data in the form of a patient data management system (PMS), which receives, in addition to diagnoses and treatment information corresponding to the patient, also data and/or measured values concerning this patient of the process gas analysis units, stores same as data sets and organizes the access thereto. The data network or bus system may also organize as a central element of the system the collaboration of the individual control units with a central control unit, so that at least some of the components of the system, for example, ventilation system, oxygenation system, dispensing system, switching unit, control units, individual control units, control modules, and process gas analysis units are connected to one another by means of the data network or bus system and can interact in a coordinated manner.

Another preferred embodiment of a data network or network linking system that is configured and intended for providing and coordinating data in the system, in the control unit or in the individual control units, in the blood gas analysis unit, in the process gas analysis units, in the ventilation system, in the oxygenation system, in the switching unit, in the dispensing system or in other components may be formed with data lines or data links, wired or wireless data network (Ethernet, LAN, WLAN, Bluetooth, PAN) or bus system (CAN, LON), data nodes for data coordination (switch, hub, router), components (database, server, router, access point) for data storage, data distribution.

In a preferred embodiment of the system, a physiological patient monitoring (PPM) system may be arranged in the system or be associated with the system. This other preferred embodiment offers the advantage that the effect of the administration of volatile substances and/or drugs and/or anesthetics on the state of the patient is monitored by measurement on the basis of physiological measured quantities, such as oxygen saturation in the blood (SPO₂), carbon dioxide concentration during and at the end of the exhalation phase (end-tidal carbon dioxide concentration, etCO₂), heart rate, blood pressure, and body temperature. The user can infer from these measured quantities the current treatment situation both of the blood-gas exchange in the lungs and of the extracorporeal blood-gas exchange with regard to the removal of carbon dioxide and to the feeding of oxygen. In addition, it is possible to use the oxygen saturation in the blood (SPO₂) as a controlled variable for the dispensing of oxygen in the dispensing system; moreover, the splitting of the fresh gas and/or possibly additional quantities of oxygen to the ventilation system and to the oxygenation system can thus also be controlled in the switching unit. The carbon dioxide concentration may be used as a basis for the control of the extracorporeal blood-gas exchange through the oxygenation system, which control may be carried out, for example, by means of adjustments of quantities to be fed to the blood feed unit and/or flow rates of the purge gas.

In a preferred embodiment of the system, a heart and lung imaging and diagnostic system may be arranged in the system or be associated with the system. This other preferred embodiment offers the advantage that the state of the lungs, and in particular also changes (improvement, recovery, exacerbation) related to the situation of the lungs during the treatment, can be followed during the treatment.

Suitable imaging systems are, for example, ultrasound diagnostic procedures, electrical impedance tomography (EIT), computer tomography (CT), X-ray, magnetic resonance imaging (MRI). In this case, especially electrical impedance tomography (EIT) can be emphasized, since - unlike the other four systems mentioned - it offers the possibility of a continuous imaging of the lungs, thorax and heart. Thus, global and/or regional changes in the state of the lungs, the type of ventilation of the lungs with possibly regional hyperdistensions and collapses can be made visible. Changes in the manner of the ventilation by the ventilation system and in the manner of the combined use with the oxygenation system for the extracorporeal blood-gas exchange are thus visible to the user in images and can be checked in terms of effect in a timely manner.

An especially preferred embodiment of the system makes possible by means of providing data an exchange of data within the system with components of the system and with the data network or network linking system. An exchange of data by the ventilation system, oxygenation system, dispensing system, switching unit, control units, process gas analysis units, blood gas analysis units, a heart and lung imaging and diagnostic system or a physiological patient monitoring (PPM) system with one another or with the data network or with the network linking system can be made possible here. Thus, the control unit of the switching unit, the control unit of the dispensing system or the individual control units in the system can be enabled to control and/or to coordinate the switching unit and/or the dispensing unit. This other preferred embodiment offers the advantage that the above-mentioned advantages of control possibilities and ability to check the effects and interactions of the ventilation system, the dispensing system, the switching unit, the oxygenation system can be provided to the user combined with one another. The data exchange makes it possible to compare and combine data with one another in the context of time and to display and document the trend of the treatment in its entirety.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a first schematic view of a system for ventilation with oxygenation and decarboxylation; and

FIG. 2 is a schematic view of a second, expanded system for ventilation with oxygenation and decarboxylation.

### DESCRIPTION OF PREFERRED EMBODIMENTS

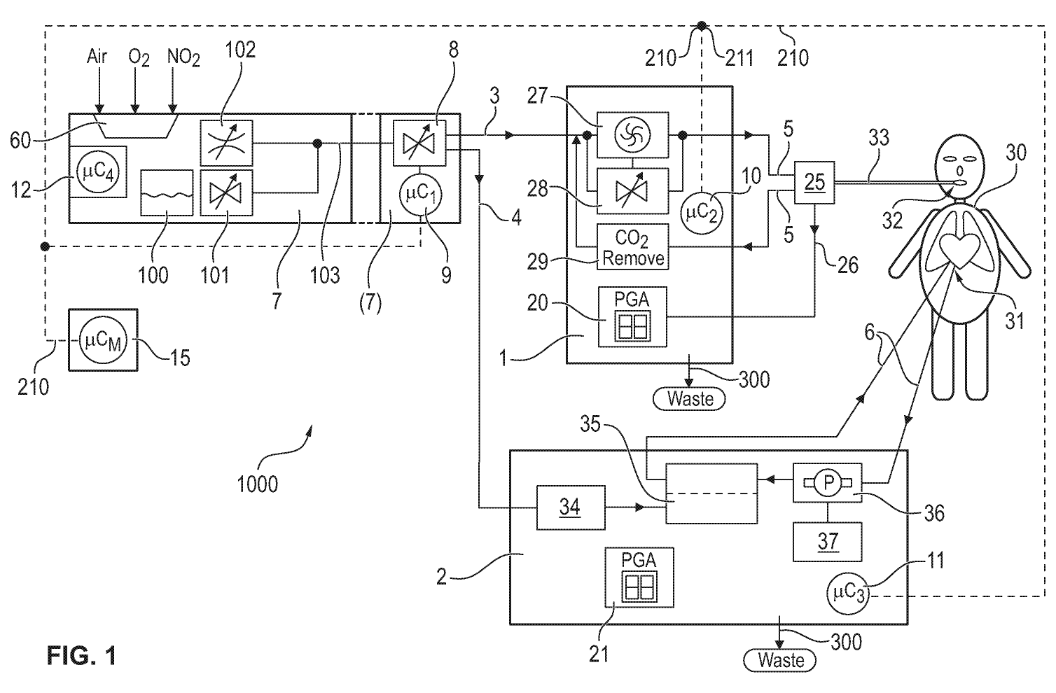

Referring to the drawings, FIG. 1 shows in a schematic view a patient 30 and a system 1000 for ventilation with oxygenation and decarboxylation with essential principal components: Ventilation system 1, oxygenation system 2, breathing gas dispensing path 3, purge gas dispensing path 4, breathing gas connection system 5, oxygenation connection system 6, dispensing system 7, switching unit 8 and a controller comprising at least one control unit 9 intended and configured for the control of the dispensing system 7. The patient is connected fluidically to the ventilation system 1 by means of the breathing gas connection system 5 for the feeding and removal of breathing gases or breathing gas mixtures. The dispensing system 7 is configured, by means of a control unit 12 of an adjusting element, for an automatic dispensing 101, e.g., in the configuration of an electronic mixer and/or active, usually electronically controlled anesthetic dispenser (electronic vaporizer) or by means of a passive anesthetic dispenser (vaporizer) 102, which may have, for example, a manually actuatable setting element (hand wheel), to dispense a quantity of volatile anesthetic into the fresh gas mixture (FG) 103 from a reservoir 100 containing volatile anesthetic. An alternative embodiment variant for a manual dispensing or mixing of gas would be an arrangement of so-called flow tubes, which can make possible a mixing of gas and/or dispensing of substances or anesthetics in interaction with needle valves and with floating body flowmeters arranged in a rising pipe. The switching unit 8 is configured by means of the control unit 9 for a distribution or splitting of the quantity of the fresh gas 103 enriched with volatile anesthetic to the ventilation system 1 or to the oxygenation system 2.

The dispensing system 7 and the switching unit 8 are shown as separate units in FIG. 1; however, the switching unit 8 may also be configured as an assembly unit (7) of the dispensing system 7 in embodiments that are used in practice.

The controller, comprising the control unit 9 and the control unit 12, may have a modular configuration or may be configured as a common control unit and may also form a central control unit 15 of the system 1000. In the dispensing system 7, the fresh gas 103 is prepared by means of a gas mixer (not shown in this schematic overview) of gases supplied by means of a gas port 60. The gases oxygen, nitrous oxide and medical air are fed to the gas port 60 - usually by means of a central gas supply unit (GS). A total quantity of enriched fresh gas 103 enters the switching unit from the dispensing system 7 and from there as a respective partial quantity via the purge gas dispensing path 4 to the oxygenation system 2 and as another partial quantity via the breathing gas dispensing path 3 to the ventilation system 1. In the ventilation system 1, a gas feed unit 27 or an alternative usable piston drive 28 is controlled by means of a control unit 10 in order to regulate the feeding of fresh gas (FG) 103 enriched with anesthetic as a breathing gas mixture to the patient 30, as well as the removal of used breathing gases or breathing gas mixtures from the patient 30 and the subsequent removal of carbon dioxide by means of an absorber unit (carbon dioxide absorber) 29 and the subsequent return to the fresh gas (FG) 103.

The breathing gas connection system 5 is comprised of an inhalation ventilation tube for the feed of the fresh gas (FG) 103 enriched with anesthetic as breathing gas mixture and an exhalation ventilation tube for the removal of the used exhaled gases or breathing gas mixtures of the patient 30, which are connected to one another for the connection of the patient 30 by means of a port and patient connection element 25 located close to the patient, a so-called Y-piece. Setting and display elements, sensors for pressure and flow rates, valves, an APL (adjustable pressure-limiting) valve, a manual ventilation bag, nonreturn valves and other components necessary for the control of the ventilation system 1 and for carrying out the ventilation are not shown in this FIG. 1 for the sake of clarity.

The patient 30 is connected to the oxygenation system 2 by means of the oxygenation connection system 6 for a supply with feeding and removal of quantities of blood into the blood circulation via an invasive fluid access 31. The patient 30 may be connected to the oxygenation system 2 via a fluid port 37, which is configured for a pumpless extracorporeal membrane oxygenation. In this case, the quantities of blood are transported towards the patient 30 and away from the patient 30 in such an embodiment by the pumping capacity of the heart of the patient themself.

This process is called pumpless extracorporeal membrane oxygenation or pumpless extracorporeal lung assist (pECLA). However, the patient 30 is connected to the oxygenation system 2 usually by means of a blood feed unit 36, which is usually configured as a pump.

The fresh gas (FG) 103 enriched with anesthetic enters a gas port 34 at the oxygenation system 2 from the switching unit 8 as purge gas by means of the purge gas dispensing path 4. By means of a control unit 11, the oxygenation system 2 controls a flow quantity and flow rate of purge gas at the membrane 35. The membrane is configured to introduce oxygen from the purge gas into the blood and to scavenge carbon dioxide from the blood into the purge gas. In this manner, a blood-to-gas exchange takes place outside of the body (extracorporeally).

The setting and display elements, sensors for pressure and flow rates, valves and other components necessary, furthermore, for the control of the oxygenation system 7 and for the carrying out of the extracorporeal enrichment with oxygen (oxygenation) and removal of carbon dioxide (decarboxylation) are not shown in this FIG. 1 for the sake of clarity.

A process gas analysis unit 20 associated with the ventilation system 1 for an analysis of the gas composition of the breathing gas or of the breathing gas mixture and another process gas analysis unit 21 associated with the oxygenation system 2 for an analysis of the gas composition of the purge gas are shown as other essential components of the system 1000. The process gas analysis units 20, 21 also have elements for display and visualization, as well as operating elements, which enable the user to read and operate, in addition to the measurement-based elements for the determination of gas concentrations. A measured gas line (sample line) 26, through which samples of the breathing gas mixture fed to the patient 30 can be fed to the process gas analysis unit 20, can be connected to the port and connection element 25 located close to the patient, so that the process gas analysis unit 20 is capable of determining by measurement the concentrations of oxygen, carbon dioxide, nitrous oxide or anesthetics and of determining and providing measured values, which indicate these concentrations.

The other process gas analysis unit 21 associated with the oxygenation system 2 is configured for an analysis of the gas composition of the purge gas. The purge gas is fed to the process gas analysis unit 21 and is analyzed in the process gas analysis unit 21 in order to monitor the ratios of carbon dioxide and oxygen at the membrane 35, so as to determine the gas exchange and the transfer rate between the blood circulation and purge gas and then to provide by means of the control unit 11 a control of oxygenation and decarboxylation that is adequate for the patient. Used gas quantities are removed from the system 1000 by the oxygenation system 2 and the ventilation system 1 via valve arrays, correspondingly provided for this purpose and not shown in FIG. 1, via a waste gas outlet (waste) 300. These used gas quantities are usually introduced from the anesthesia device into the infrastructure of the hospital by means of an anesthesia gas scavenging system and then disposed of therein correspondingly in a timely manner. Depending on the splitting of the fresh gas (FG) 103 in the breathing circuit or in the blood circulation, the carrying out of the anesthesia with substances, preferably volatile substances, especially anesthetics, takes place by inhalation with the ventilation system 1 at the same time as the carrying out of the ventilation with a gas-to-blood exchange in the lungs of the patient 30 or extracorporeally with a gas-to-blood exchange at the membrane 35 of the oxygenation system 2.

The ratio of the administration of anesthetics by inhalation and extracorporeally and thus the ratio of the anesthesia produced by inhalation to the extracorporeal anesthesia can be set for the user via the switching unit 8. The measured values of the process gas analysis unit (PGA) 20 of the ventilation system, as well as the measured values and status values of the process gas analysis unit (PGA) 21 of the oxygenation system 2 are available to the user as support.

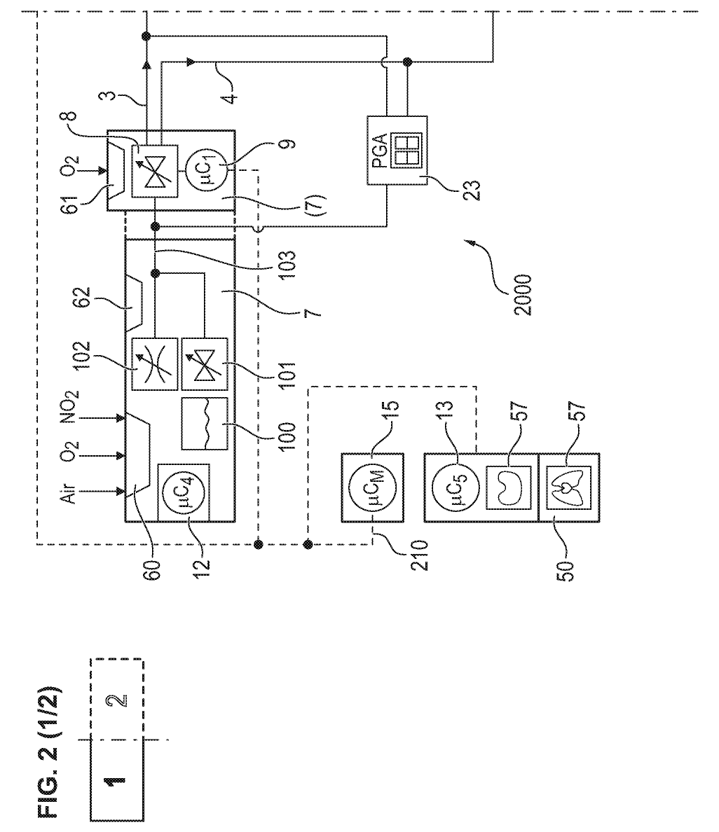

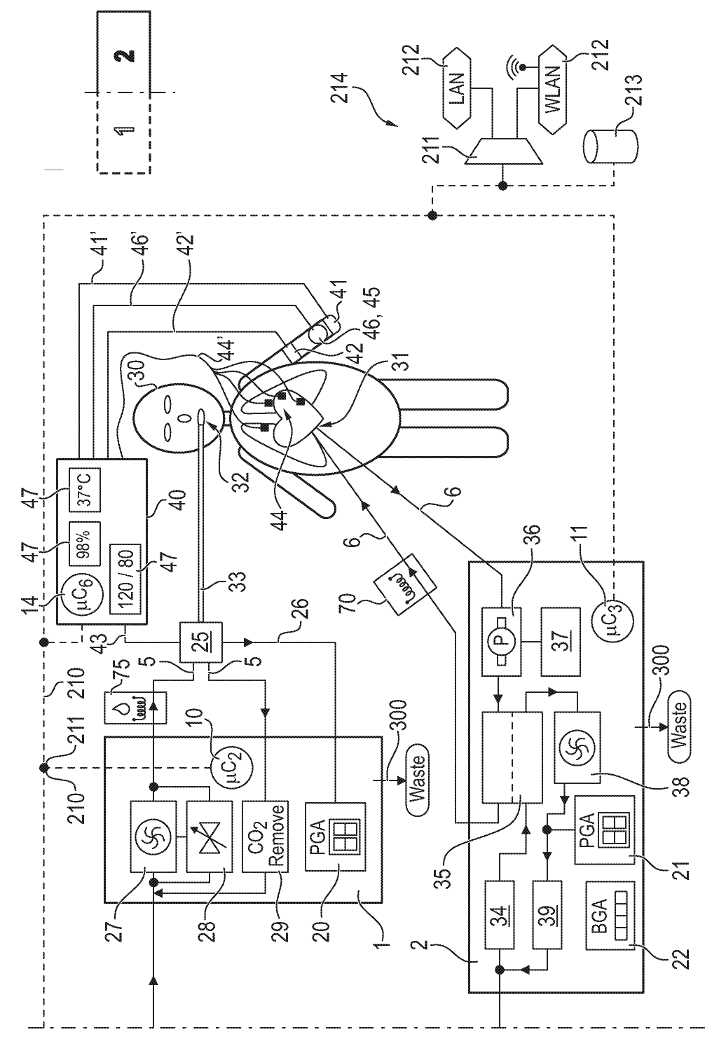

Data interfaces, which can make possible the unidirectional and/or bidirectional exchange of data between the ventilation system 1, the oxygenation system 2, the dispensing system 7, and the switching unit 8, may be provided at the ventilation system 1, at the oxygenation system 2, at the dispensing system 7, and at the switching unit 8. Such an exchange of data is preferably organized, initiated or coordinated in interaction and communication with the control units 9, 10, 11, 12 in the ventilation system 1, the oxygenation system 2, the dispensing system 7, and the switching unit 8. The data interfaces are connected to one another in a wired or wireless manner by means of data lines 210 (FIG. 2) and data nodes 211 (FIG. 2). An additional central control unit 15 (FIG. 2), not shown in this FIG. 1, may also be arranged in the system 1000, as well as in the system 2000 (FIG. 2) and intended to coordinate via data lines 210 (FIG. 2) the interaction of the ventilation system 1, the oxygenation system 2, the dispensing system 7, and the switching unit 8 in the system 1000, and possibly also with other components 210, 211, 212, 213, 214 (FIG. 2) (database, server, router, access point, hub) in a data network 212 (FIG. 2) (LAN, WLAN, Bluetooth, PAN, Ethernet) or network linking system (214).

FIG. 2 shows with a system 2000 additional possibilities for expansions and embodiments of the system 1000 according to the present invention for ventilation with oxygenation and decarboxylation according to FIG. 1.

Identical components in FIG. 1 and in FIG. 2 are designated with the same reference numbers in FIGS. 1 and 2.

In addition to the elements and components shown in FIG. 1 and described in regard to the system 1000 (FIG. 1) 1, 2, 3, 4, 5, 6, 7, 8, 9, 10, 11, 12, 15, 21, 25, 26, 27, 28, 30, 31, 32, 33, 34, 35, 36, 37, 100, 101, 102, 103, 210, 211, 300, other features and components 13, 14, 22, 23, 38, 39, 40, 41, 42, 43, 44, 45, 46, 47, 50, 57, 61, 212, 213, 214 are present in the expanded system 2000 according to FIG. 2.

Thus, the expanded system 2000 has optional additional gas ports 61, 62, an additional gas feed unit (blower) 38 and an additional absorber unit (carbon dioxide absorber) 39 in the oxygenation system 2. Such an additional gas feed unit 38 may be arranged in the oxygenation system 2 combined with the other absorber unit 39 as a module, for example, as a type of plug-in module. The additional gas feed unit 38, as well as the additional absorber unit 39, may be configured together or separately also as independent units or modules, which can be connected to the oxygenation system 2, for example, as external modules.

The expanded system 2000 thus includes a physiological patient monitoring system 40. The physiological patient monitoring system 40 has displays 47 and visualizations of acquired, determined, analyzed or calculated physiological measured data and/or parameters. These include, for example, measurement-based determinations of ECG 44 by means of ECG electrodes on the upper body of the patient 30 and ECG cable 44', detection of an oxygen saturation (SPO₂) 41', for example, on a finger 41 of the patient 30, detection of a non-invasive blood pressure measured value 42' by means of a blood pressure cuff 42 on the upper arm of the patient 30, detection of an invasive blood pressure measured value 46' by means of an invasive access point on the hand 46 of the patient 30, as well as of a body temperature, for example, of a skin temperature or of a body core temperature of the patient 30.

Via an optional port for gas suction at the Y-piece 25 and an additional measured gas line 43, gas samples can be sent to the physiological patient monitoring system 40 and gas analyses can be carried out therein, for example, concentrations of carbon dioxide, methane or analyses on other components, for example, alcohols (ethanol) of the exhaled gas or breathing gas mixture.

The expanded system 2000 has a heart and lung imaging and diagnostic system 50. Heart and lung imaging and diagnostic systems 50 are configured, for example, as devices for computer tomography (CT diagnostics), magnetic resonance imaging (MRI diagnostics), X-ray devices (X-ray diagnostics), devices for electrical impedance tomography (EIT diagnostics) or devices for ultrasound diagnostics (US diagnostic sonography, Doppler sonography). The heart and lung imaging and diagnostic system 50 can provide to the user valuable information about what state of disease or recovery the lungs of the patient 30 are in. Based on this, the user can configure the system 2000 to that effect in order to place the focus of the feed of oxygen to the patient 30 by inhalation on the path via the lungs or by means of the extracorporeal membrane oxygenation invasively on the path via the blood circulation. Unlike CT diagnostics, X-ray diagnostics, MRI diagnostics, US diagnostics, electrical impedance tomography devices 50 (EIT diagnostics) especially make possible a continuous imaging of the lungs, thorax and heart. Possible changes in the state of the lungs can thus be made visible with the EIT diagnostics continuously and in a timely manner during the treatment.

Effects of the ventilation and of the manner of the combined use with the oxygenation system are thus visible and can be checked in a timely manner. In addition, in the expanded system 2000 it may have an additional process gas analysis unit (PGA) 23, for example, arranged at the switching unit 8 or at the dispensing unit 7 for the analysis of the fresh gas (FG) 103 in the oxygenation dispensing path and/or in the breathing gas dispensing path 3, the purge gas dispensing path 4 or starting from the dispensing unit 7. Thus, information with regard to the dispensing and setting of the anesthetic vaporizer 101, 102 can thus be checked by means of measurement-based concentration determination in the fresh gas 103. Depending on the set splitting of the fresh gas (FG) 103 in the breathing circuit or in the blood circulation and depending on the quantity of additional feeding of oxygen at the additional gas port 61 to the switching unit 8, the gas in the breathing gas dispensing path 3 and the gas in the purge gas dispensing path 4 have different oxygen concentrations. The additional process gas analysis unit (PGA) 23 may be useful to monitor this difference based on measurement. In such a mode of operation, the anesthesia is carried out with the ventilation system 1 with the feeding of volatile anesthetic as well as with the feeding of other substances, preferably volatile substances, by inhalation at the same time as the ventilation is carried out with a gas-to-blood exchange at the membrane 35 of the oxygenation system 2, as desired by the user, with different concentrations of oxygen in the breathing gas or breathing gas mixture indirectly 5, 32, 33 to the lungs of the patient 30 and indirectly 6, 31 into the blood circulation of the patient 30.

For further analysis, the expanded system 2000 may have, in addition, a blood gas analysis unit (BGA) 22 for the analysis of blood gases in the blood of the patient 30 in the oxygenation system 2. A blood gas analysis provides, for example, information with regard to a gas distribution (partial pressure) of O₂ (oxygen), CO₂ (carbon dioxide) as well as the pH value and the acid-base balance in the blood of the patient 30. Such a blood gas analysis unit 22 (BGA) may be arranged combined with the process gas analysis unit (PGA) 21 as a module, for example, as a type of plug-in module in the oxygenation system 2. The blood gas analysis unit (BGA) 22 and the process gas analysis unit (PGA) 21 may be configured together or separately also as independent units or modules, which can be connected to the oxygenation system 2, for example, as external modules.

In addition to dispensing of volatile anesthetics 100 by means of anesthetic evaporation 101, 102, feeding of other substances, for example, drugs by drug nebulization, feeding of other gases or gas mixtures, for example, nitrogen monoxide, helium, Heliox may be carried out by means of the additional gas port 62 at the dispensing system 7.

The systems 1000, 2000 shown in FIGS. 1 and 2 may be connected to the other medical devices or systems, for example, to process gas analysis units 20, 21, 23, blood gas analysis units (BGA) 22, to the physiological patient monitoring (PPM) system 40 as well as to the heart and lung imaging and diagnostic system 50 for an interaction and for a common system operation by means of the data nodes 211, data lines 210 in the data network 212.

While specific embodiments of the invention have been shown and described in detail to illustrate the application of the principles of the invention, it will be understood that the invention may be embodied otherwise without departing from such principles.

### List of Reference Numbers

1 Ventilation system
2 Oxygenation system (oxygenator)
3 Breathing gas dispensing path
4 Purge gas dispensing path
5 Breathing gas connection system
6 Oxygenation connection system
7 Dispensing system
8 Switching unit
9 Control unit, control module (μC1) of the switching unit
10 Control unit, control module (μC2) of the ventilation system
11 Control unit, control module (μC3) of the oxygenation system
12 Control unit, control module (μC4) of the dispensing system
13 Control unit, control module (μC5) of the imaging system
14 Control unit, control module (μC6) of the physiological patient monitoring (PPM) system
15 External control unit, external control module (μCM)
20 Process gas analysis (PGA, PGA-VS) of the ventilation system
21 Process gas analysis (PGA, PGA-OS) of the oxygenation system
22 Blood gas analysis (BGA) of the oxygenation system
23 Process gas analysis (PGA, PGA-DS) at the dispensing system/switching unit
25 Port and connection element (Y-piece) of the breathing gas connection system located close to the patient
26 Measured gas line for connection of the port and connection element located close to the patient to the process gas analysis of the ventilation system
27 Gas feed unit (blower) in the ventilation system
28 Alternative gas feed unit (piston drive) in the ventilation system
29 Absorber unit, carbon dioxide absorber (CO₂ remove) in the ventilation system
30 Patient, living being
31 Invasive fluid access to the blood circulation of the patient
32 Access to the airways of the patient
33 Endotracheal tube, alternative nasal mask or tracheostoma
34 Gas port at the oxygenation system
35 Membrane, blood<->gas exchange membrane, oxygenator membrane
36 Fluid port with blood feed unit (pump) for feeding quantities of blood between the patient and the membrane
37 Fluid port in case of pumpless extracorporeal membrane oxygenation
38 Gas port with additional gas feed unit (blower) in or at the oxygenation system, (possibly as plug-in module)
39 Absorber unit, carbon dioxide absorber (CO₂ remove) in the oxygenation system
40 Physiological patient monitoring (PPM) system
41 Oxygen saturation (SPO₂) measuring point (hand, finger)
41' Oxygen saturation (SPO₂) measuring line
42 Non-invasive blood pressure (NIBP) measurement, blood pressure cuff
42' Connecting line to the blood pressure cuff
43Port for process gas analysis of breathing gases of the patient
44 ECG measurement, arrangement of ECG electrodes on the patient
44' ECG connection cable
45 Invasive blood pressure (IBP) measurement
46 Invasive access point (back of hand) at the patient
46' Invasive access line
47 Display of physiological measured values (NIBP, IBP, ECG, CO₂, SPO₂, HR, temperature) in the form of numerical values, diagrams, graphs
50 Heart and lung imaging and diagnostic system (EIT, CT, MRI, X-ray, ultrasound)
57 Display of images, diagrams, graphs, numerical values
60 Gas port for feeding oxygen, nitrous oxide, air to the dispensing system
61 Additional gas port at the switching unit
62 Additional gas port at the dispensing system for feeding an additional other gas, e.g., oxygen to the dispensing system
70 Heating system for quantities of blood at the oxygenation connection system
75 Humidification and/or heating system for breathing as at the breathing gas connection system
100 Reservoir (tank) containing volatile substances, volatile anesthetics
101 Anesthetic dispenser (vaporizer) with adjusting element for the automated feed of quantities of volatile anesthetics into the fresh gas (FG) mixture
102 Anesthetic dispenser (vaporizer) with manually actuatable setting element (hand wheel) for dispensing of volatile anesthetics into the fresh gas (FG) mixture
103 Supply of the fresh gas mixture to the switching unit 8
210 Data lines, data links, data nodes
211 Data nodes, data coordination (switch, hub, router)
212 Data network (LAN, WLAN, Bluetooth, PAN, Ethernet)
213 Components in the data network (database, server, router, access point, hub)
214 Network linking system
300 Waste gas outlet (waste)
1000 System (FIG. 1)
2000 Expanded system (FIG. 2)

What is claimed is:

1. A system for ventilating and oxygenating a patient, the system comprising:
a ventilation system with a breathing gas dispensing path, the ventilation system being configured with devices to supply, feed and remove breathing gas mixtures and substances to and from the patient;
an oxygenation system with a purge gas dispensing path;
a breathing gas connection system connected to the ventilation system, the breathing gas connection system being configured for a gas-carrying connection for supplying, feeding and removing quantities of breathing gas mixtures and substances to and from the patient;
an oxygenation connection system connected to the oxygenation system;
a dispensing system configured for a dispensing of one or more substances;
a switching unit connected to the dispensing system and configured to switch the dispensing of substances of the dispensing system between the breathing gas dispensing path and the purge gas dispensing path wherein:
the purge gas dispensing path is configured for connection of the oxygenation system to the dispensing system or to the switching unit and a quantity of a fresh gas mixture, enriched with a quantity of the one or more substance, is supplied as a purge gas from the dispensing system and the switching unit to the oxygenation system by means of the purge gas dispensing path;
the oxygenation system comprises a membrane for a gas exchange with a blood circulation of the patient with a feeding of a quantity of oxygen and of a quantity of a volatile substance of the one or more substances into the blood circulation of the patient and a removal of carbon dioxide from the blood circulation of the patient, the oxygenation system comprising devices configured to feed and/or supplying a quantity of the purge gas to the membrane and to supply the patient with quantities of blood enriched with the volatile substance and remove carbon dioxide from the blood circulation of the patient, wherein the purge gas is configured to receive the carbon dioxide from the blood circulation of the patient at the membrane; and
the breathing gas dispensing path is configured for connection of the ventilation system to the dispensing system or to the switching unit and a quantity of a fresh gas mixture enriched with a quantity of a volatile substance of the one or more substances is supplied as a breathing gas mixture from the dispensing system and the switching unit to the ventilation system by means of the breathing gas dispensing path; and
a controller, comprising at least one control unit, configured to control the switching unit; and
a gas feed unit arranged in the purge gas dispensing path and/or in the oxygenation system for a transport of gas, wherein an absorber unit is arranged in the purge gas dispensing path and/or in the oxygenation system for a removal of carbon dioxide from the purge gas, wherein the controller is configured as a central control system or as a central control unit, the controller comprising individual control units and the at least one control unit being one of the individual control units forming a non-central control system, the at least one control unit being arranged as a part of the oxygenation system and/or as a part of the ventilation system.

2. A system in accordance with claim 1, wherein the dispensing system is configured for a dispensing of volatile substances and/or for a dispensing of volatile anesthetics as the one or more substance.

3. A system in accordance with claim 1, further comprising a blood feed unit arranged in or at the oxygenation connection system and/or at the oxygenation system for transport of quantities of blood to the patient and/or away from the patient.

4. A system in accordance with claim 1, wherein:
the controller comprises individual control units and the at least one control unit is one of the individual control units forming a non-central control system; and
the non-central control system comprises the at least one control unit as a single control unit in the switching unit and/or a single control unit in the dispensing system and/or an external control unit;
wherein one of the single control units and/or the external control unit is configured for a control of the switching unit and/or of the dispensing system.

5. A system in accordance with claim 4, wherein at least one of the control units takes into consideration data provided from the respective ventilation system and/or the oxygenation system in controlling the switching unit.

6. A system in accordance with claim 1, further comprising a process gas analysis unit arranged in or at the ventilation system or in or at the breathing gas connection system or associated with the ventilation system or with the breathing gas connection system and configured for an analysis, wherein the process gas analysis unit is configured to provide data determined on the basis of the analysis to the ventilation system to the system and/or to the at least one control unit.

7. A system in accordance with claim 1, further comprising a process gas analysis unit arranged in or at the oxygenation system, arranged in or at the oxygenation connection system, associated with the oxygenation system, or arranged in or at the oxygenation connection system, wherein the process gas analysis unit is configured to provide analysis data to the system and/or to the at least one control unit.

8. A system in accordance with claim 1, further comprising a central process gas analysis unit, wherein the central process gas analysis together with a switching unit and distribution control unit is configured to carry out analyses of gas samples of the ventilation system, of the breathing gas connection system, of the oxygenation system, of the oxygenation connection system, of the dispensing system or of the switching unit and to provide analysis data determined on the basis of the analysis to the system and/or to the at least one control unit.

9. A system in accordance with claim 1, further comprising a blood gas analysis unit arranged in or at the oxygenation system or in or at the oxygenation connection system or associated with the oxygenation system or with the oxygenation connection system for an analysis, wherein the blood gas analysis unit is configured to provide data determined on the basis of the analysis to the oxygenation system to the system and/or to the at least one control unit.

10. A system in accordance with claim 9, further comprising a process gas analysis unit arranged in or at the switching unit or in or at the dispensing system or associated with the switching unit or with the dispensing system for an analysis, wherein the process gas analysis unit is configured to provide analysis data determined based on analysis to the switching unit, to the dispensing system, to the system and/or to the at least one of the control unit.

11. A system in accordance with claim 1, further comprising an absorber unit arranged in the breathing gas connection system and/or in the ventilation system for a removal of carbon dioxide from the breathing gases.

12. A system in accordance with claim 11, further comprising a data network arranged in or at the system or associated with the system, wherein the data network is configured to provide data to the system, to the at least one control unit, to the ventilation system, to the oxygenation system, to the switching unit, or to the dispensing system to enable therewith the controller to control and/or coordinate the switching unit and/or the dispensing unit.

13. A system in accordance with claim 12, further comprising a physiological patient monitoring system configured to provide data to the system, to the ventilation system, to the oxygenation system, to the dispensing system or to the switching unit and/or to the at least one control unit.

14. A system in accordance with claim 1, further comprising a heart and lung imaging and diagnostic system configured to provide data to the system, to the ventilation system, to the oxygenation system, to the dispensing system, to the switching unit and/or to the at least one control unit.

15. A system in accordance with claim 12, wherein the system is configured to provide data in a data exchange with the data network.

16. A system for ventilating and oxygenating a patient, the system comprising:
a ventilation system with a breathing gas dispensing path, the ventilation system being configured with devices to supply, feed and remove breathing gas mixtures and substances to and from the patient;
an oxygenation system with a purge gas dispensing path;
a breathing gas connection system connected to the ventilation system, the breathing gas connection system being configured for a gas-carrying connection for supplying, feeding and removing quantities of breathing gas mixtures and substances to and from the patient;
an oxygenation connection system connected to the oxygenation system;
a dispensing system configured for a dispensing of one or more substances;
a switching unit connected to the dispensing system and configured to switch the dispensing of substances of the dispensing system between the breathing gas dispensing path and the purge gas dispensing path wherein:
the purge gas dispensing path is configured for connection of the oxygenation system to the dispensing system or to the switching unit and a quantity of a fresh gas mixture, enriched with a quantity of the one or more substance, is supplied as a purge gas from the dispensing system and the switching unit to the oxygenation system by means of the purge gas dispensing path;
the oxygenation system comprises a membrane for a gas exchange with a blood circulation of the patient with a feeding of a quantity of oxygen and of a quantity of a volatile substance of the one or more substances into the blood circulation of the patient and a removal of carbon dioxide from the blood circulation of the patient, the oxygenation system comprising devices to supply the patient with quantities of blood enriched with the volatile substance and to remove carbon dioxide from the blood circulation of the patient, the membrane defining a volatile substance and carbon dioxide transfer interface, wherein the purge gas is configured to receive the carbon dioxide from the blood circulation at the membrane and the purge gas is configured to provide oxygen to the blood circulation at the membrane; and
the breathing gas dispensing path is configured for connection of the ventilation system to the dispensing system or to the switching unit and a quantity of a fresh gas mixture enriched with a quantity of a volatile substance of the one or more substances is supplied as a breathing gas mixture from the dispensing system and the switching unit to the ventilation system by means of the breathing gas dispensing path; and
a controller, comprising at least one control unit, configured to control the switching unit;
a gas feed unit arranged in the purge gas dispensing path and/or in the oxygenation system for a transport of gas, wherein an absorber unit is arranged in the purge gas dispensing path and/or in the oxygenation system for a removal of carbon dioxide from the purge gas, wherein the controller is configured as a central control system or as a central control unit, the controller comprising individual control units and the at least one control unit being one of the individual control units forming a non-central control system, the at least one control unit being arranged as a part of the oxygenation system and/or as a part of the ventilation system.

17. A system for ventilating and oxygenating a patient, the system comprising:
a ventilation system with a breathing gas dispensing path, the ventilation system being configured with devices to supply, feed and remove breathing gas mixtures and substances to and from the patient;
an oxygenation system with a purge gas dispensing path;
a breathing gas connection system connected to the ventilation system, the breathing gas connection system being configured for a gas-carrying connection for supplying, feeding and removing quantities of breathing gas mixtures and substances to and from the patient;
an oxygenation connection system connected to the oxygenation system;
a dispensing system configured for a dispensing of one or more substances;
a switching unit connected to the dispensing system on an inlet side of the switching unit, the switching unit being connected to the purge gas dispensing path and the ventilation system on an outlet side of the switching unit, the switching unit being configured to switch the dispensing of substances of the dispensing system between the breathing gas dispensing path and the purge gas dispensing path wherein:
the purge gas dispensing path is configured for connection of the oxygenation system to the dispensing system or to the switching unit and a quantity of a fresh gas mixture, enriched with a quantity of the one or more substance, is supplied as a purge gas from the dispensing system and the switching unit to the oxygenation system by means of the purge gas dispensing path;
the oxygenation system comprises a membrane for a gas exchange with a blood circulation of the patient with a feeding of a quantity of oxygen and of a quantity of a volatile substance of the one or more substances into the blood circulation of the patient and a removal of carbon dioxide from the blood circulation of the patient, the oxygenation system comprising devices configured to feed and/or supplying a quantity of the purge gas to the membrane and to supply the patient with quantities of blood enriched with the volatile substance and remove carbon dioxide from the blood circulation of the patient, wherein the purge gas is configured to receive the carbon dioxide from the blood circulation of the patient at the membrane; and
the breathing gas dispensing path is configured for connection of the ventilation system to the dispensing system or to the switching unit and a quantity of a fresh gas mixture enriched with a quantity of a volatile substance of the one or more substances is supplied as a breathing gas mixture from the dispensing system and the switching unit to the ventilation system by means of the breathing gas dispensing path; and
a controller, comprising at least one control unit, configured to control the switching unit for delivering the purge gas from the dispensing system and the switching unit to the oxygenation system via the purge gas dispensing path in a first position of the switching unit, and to control the switching unit for delivering the quantity of the fresh gas mixture enriched with a quantity of the volatile substance of the one or more substances from the dispensing system and the switching to the ventilation system via the breathing gas dispensing path in a second position of the switching unit.

18. A system in accordance with claim 17, further comprising a gas feed unit arranged in the purge gas dispensing path and/or in the oxygenation system for a transport of gas, wherein an absorber unit is arranged in the purge gas dispensing path and/or in the oxygenation system for a removal of carbon dioxide from the purge gas.

19. A system in accordance with claim 18, wherein the controller is configured as a central control system or as a central control unit, wherein the controller comprises individual control units and the at least one control unit is one of the individual control units forming a non-central control system, wherein the at least one control unit is arranged as a part of the oxygenation system and/or as a part of the ventilation system.

20. A system in accordance with claim 19, wherein the central control system or the central control using is configured to coordinate an interaction of the ventilation system, the oxygenation system, the dispensing system, and the switching unit based on operating data received from the ventilation system and the oxygenation system.
